(19) United States
(12) Patent Application Publication
(10) Pub. No.: US 2024/0374257 A1
(43) Pub. Date: Nov. 14, 2024

(54) SYSTEMS AND METHODS FOR PROVIDING A VACUUM ASSISTED SECONDARY CLOSURE DEVICE

(71) Applicant: Michael N. Desvigne, Peoria, AZ (US)

(72) Inventor: Michael N. Desvigne, Peoria, AZ (US)

(21) Appl. No.: 18/657,603

(22) Filed: May 7, 2024

Related U.S. Application Data
(60) Provisional application No. 63/464,892, filed on May 8, 2023.

(51) Int. Cl. A61B 17/08; A61B 17/00; A61F 13/0246; A61F 13/05; A61L 15/40; A61L 15/64

(52) U.S. Cl. CPC A61B 17/085; A61F 13/025; A61F 13/05; A61L 15/40; A61L 15/64; A61B 2017/00004; A61B 2017/081

(57) ABSTRACT

While the described biocompatible wound dressing can include any suitable component, in some cases, it includes a biocompatible interface and multiple fixation elements that are each configured to at least one of absorb and incorporate into a user's body after prolonged use in the user's body. In some cases, the interface has a first surface and a second surface that is disposed substantially opposite to the first surface, with a peripheral edge that extends around an outer perimeter of the biocompatible interface. In some cases, the fixation elements extend from the first surface of the interface, adjacent to the peripheral edge. In some cases, the fixation elements also have a hook-like shape that is configured to couple the interface to subcutaneous tissue on an undersurface of skin when the peripheral edge and the fixation elements are inserted below the subcutaneous tissue. Additional implementations are described herein.

### CROSS-REFERENCE TO RELATED APPLICATION

This application claims priority to U.S. Provisional Application Ser. No. 63/464,892 (Attorney Docket No. 30965.2), which is entitled SYSTEMS AND METHODS FOR PROVIDING A VACUUM ASSISTED SECONDARY CLOSURE DEVICE, and which was filed on May 8, 2023; the entire disclosure of which is incorporated herein by reference.

### FIELD

The described systems and methods relate to the treatment of wounds. In particular, the described systems and methods relate to a biocompatible wound dressing that can include any suitable component. In some cases, it includes one or more biocompatible interfaces and one or more fixation elements (e.g., barbs, hooks, adhesives, catches, and/or any other suitable fixation elements) that are each configured to be absorbed and/or incorporated into a user's body after prolonged use in the user's body. In some cases, the interface has a first surface and a second surface that is disposed substantially opposite to the first surface, with a peripheral edge that extends around an outer perimeter of the biocompatible interface. In some cases, the fixation elements extend from (and/or are disposed at) the first surface of the interface, adjacent to the peripheral edge. In some cases, the fixation elements also have a hook-like shape that is configured to catch or otherwise couple the interface to subcutaneous tissue on an undersurface of skin when the peripheral edge and the fixation elements are inserted below the subcutaneous tissue. In some cases, the wound dressing is used with a negative pressure wound therapy device and/or any other suitable mechanical closure device.

### Background and Related Art

Most wounds can be treated and healed through the use of stitches and/or a bandage. In some cases, however, chronic or acute wounds (such as open abdominal wounds, dehisced surgical wounds, pressure injuries, pressure ulcers, diabetic ulcers, open fractures, skin grafts, second- and third-degree burns, sternal wounds, fasciotomy wounds, and a wide variety of other wounds) may not readily heal with just stitches and/or a bandage or may benefit from planned delayed surgical closure. Thus, in some cases, in an effort to help wounds heal, medical practitioners may debride the wound, apply compression therapy to the wound, perform significant surgical procedures on the wound, apply negative pressure wound therapy to the wound, and/or otherwise treat the wound to help it heal. In this regard, negative pressure wound therapy (or vacuum assisted closure) often includes using a sealed dressing that covers the wound. Generally, the sealed dressing is attached to tubing that, in turn, is attached to a pump (or vacuum). In some such cases, negative pressure wound therapy can remove excess exudate and promote wound healing. Indeed, in some cases, negative pressure wound therapy can help increase blood flow to a wound.

While there are a variety of known techniques for treating chronic or acute wounds, and while many such techniques can be relatively effective, such techniques are not necessarily without their shortcomings. Indeed, in some cases, some methods for treating chronic or acute wounds may: be ineffective on some wounds, not work well with large wounds, take longer that desired to be efficacious, require the wound to be opened on a regular basis, require one or more materials to be placed in and to then be removed from the wound (which can be painful and cumbersome), and/or otherwise have a variety of drawbacks.

Thus, while techniques currently exist that are used to help heal or otherwise treat chronic or acute wounds, challenges still exist, including those listed above. Accordingly, it would be an improvement in the art to augment or even replace current techniques with other techniques.

### SUMMARY

The described systems and methods relate to the treatment of wounds. In particular, the described systems and methods relate to a biocompatible wound dressing that can include any suitable component. In some cases, it includes one or more biocompatible interfaces and one or more fixation elements (e.g., barbs, hooks, adhesives, catches, and/or any other suitable fixation elements) that are each configured to be absorbed and/or incorporated into a user's body after prolonged use in the user's body. In some cases, the interface has a first surface and a second surface that is disposed substantially opposite to the first surface, with a peripheral edge that extends around an outer perimeter of the biocompatible interface. In some cases, the fixation elements extend from (and/or are disposed at) the first surface of the interface, adjacent to the peripheral edge. In some cases, the fixation elements also have a hook-like shape that is configured to catch or otherwise couple the interface to subcutaneous tissue on an undersurface of skin when the peripheral edge and the fixation elements are inserted below the subcutaneous tissue. In some cases, the wound dressing is used with a negative pressure wound therapy device and/or any other suitable mechanical closure device.

While the described systems and methods may be particularly useful in the area of treating wounds in skin, the described systems and methods can be modified (e.g., by changing the material, absorbability, size, shape, and/or one or more characteristics of the biocompatible interface and/or of the fixation elements) to allow the biocompatible wound dressing to be used on bones, tendons, tissues, organs, muscles, dermis, subcutaneous facia, fatty tissue, muscle facia, facia, any musculoskeletal soft tissue deficit, and/or any other suitable portion of a patient's body. Indeed, in some cases (including, without limitation, in some embodiments in which the interface is configured to be bioabsorbable and/or to serve as a tissue scaffolding utilizing human cadaver tissue and/or ovine extracellular matrix), the described systems and methods are used to cover one or more organs or vascular structures.

These and other features and advantages of the described systems and methods will be set forth or will become more fully apparent in the description that follows and in the appended claims. The features and advantages may be realized and obtained by means of the instruments and combinations particularly pointed out in the appended claims. Furthermore, the features and advantages of the described systems and methods may be learned by the practice thereof or will be obvious from the description, as set forth hereinafter.

### BRIEF DESCRIPTION OF THE DRAWINGS

In order that the manner in which the above recited and other features and advantages of the described systems and methods are obtained, a more particular description of the described systems and methods will be rendered by reference to specific embodiments thereof, which are illustrated in the appended drawings. Understanding that the drawings depict only representative embodiments of the described systems and methods and are not, therefore, to be considered as being limiting in scope, the described systems and methods will be set forth and explained with additional specificity and detail through the use of the accompanying drawings in which:

FIG. 1 illustrates a plan view of a biocompatible wound dressing disposed in a wound in accordance with a representative embodiment;

FIG. 2 illustrates a plan view of a frontside of a representative embodiment of the biocompatible wound dressing;

FIG. 3 illustrates a plan view of a frontside of a representative embodiment of the biocompatible wound dressing;

FIGS. 4A-5A each illustrate an elevation view of different representative embodiments of the biocompatible wound dressing;

FIG. 5B illustrates an elevation view of various fixation elements in accordance with some embodiments;

FIG. 6 illustrates an elevation view of the biocompatible wound dressing in accordance with some embodiments;

FIGS. 7-10 each illustrate a plan view of the biocompatible wound dressing disposed within a wound in accordance with some embodiments;

FIG. 11 illustrates a plan view of a wound, wherein the wound has substantially healed, in accordance with a representative embodiment;

FIG. 12 illustrates a plan view of a wound, wherein the wound has substantially healed, and wherein the wound has an external bandage on it, in accordance with a representative embodiment;

FIG. 13 illustrates a plan view of the wound dressing disposed in a wound and being used with one or more sensors in accordance with a representative embodiment;

FIG. 14 illustrates a representative embodiment of a method for using the biocompatible wound dressing;

FIG. 15 illustrates an elevation view of the biocompatible wound dressing in accordance with a representative embodiment;

FIG. 16A illustrates an elevation view of the biocompatible wound dressing in accordance with a representative embodiment;

FIG. 16B illustrates an elevation view of the biocompatible wound dressing in accordance with a representative embodiment;

FIG. 17 illustrates a representative system that provides a suitable operating environment for use with some embodiments of the described biocompatible wound dressing; and

FIG. 18 illustrates a representative embodiment of a networked system that provides a suitable operating environment for use with some embodiments of the described wound dressing.

### DETAILED DESCRIPTION

The described systems and methods relate to the treatment of wounds. In particular, the described systems and methods relate to a biocompatible wound dressing that can include any suitable component. In some cases, it includes one or more biocompatible interfaces and one or more fixation elements (e.g., barbs, hooks, adhesives, catches, and/or any other suitable fixation elements) that are each configured to be absorbed and/or incorporated into a user's body after prolonged use in the user's body. In some cases, the interface has a first surface and a second surface that is disposed substantially opposite to the first surface, with a peripheral edge that extends around an outer perimeter of the biocompatible interface. In some cases, the fixation elements extend from (and/or are disposed at) the first surface of the interface, adjacent to the peripheral edge. In some cases, the fixation elements also have a hook-like shape that is configured to catch or otherwise couple the interface to subcutaneous tissue on an undersurface of skin when the peripheral edge and the fixation elements are inserted below the subcutaneous tissue. In some cases, the wound dressing is used with a negative pressure wound therapy device and/or any other suitable mechanical closure device.

As used herein, the term biocompatible and variations thereof may be used to refer to a material that is capable of being in contact with living tissue (including tissue that would generally be covered by skin) for a prolonged period of time without causing harm to such tissue.

As used herein, the term absorb and variations thereof may be used to refer to the act of a material breaking down over a period of time and passing into a body of the user.

As used herein, the term incorporate and variations thereof may be used to refer to the act of having at least a portion of a material be introduced into or be used as a scaffolding for a portion of a body, tissue, or mass, as an integral part thereof.

As used herein, the term wound and variations thereof may refer to one or more incisions, lacerations, abrasions, avulsions, amputations, puncture wounds, contusions, combinations, penetration wounds, surgical wounds, divisions of tissue, ruptures of the integument and/or mucous membrane, ulcers, diabetic ulcers, and/or any other sores in which a patient's (or user's) skin has an undesirable opening in it.

The following disclosure is grouped into two subheadings, namely “BIOCOMPATIBLE WOUND DRESSING” and “REPRESENTATIVE OPERATING ENVIRONMENT”. The utilization of the subheadings is for the convenience of the reader only and is not to be construed as limiting in any sense.

### Biocompatible Wound Dressing

In general, the described systems and methods relate to a biocompatible wound dressing that is configured to be placed in a wound, with one or more edges of a biocompatible interface of the wound dressing being disposed below a piece of tissue (e.g., below a subcutaneous layer of skin) surrounding the wound, with one or more fixation elements (e.g., hooks, barbs, catches, adhesives, or other fixation elements) coupling a first side (or surface) of the biocompatible interface to an underside of the piece of tissue, and with the biocompatible interface and/or the fixation elements being configured to be at least one of absorbed and/or incorporated into a body of a patient after prolonged use.

While the described wound dressing can perform any suitable function, in some embodiments, it is placed into a wound to: act as an occlusive dressing (e.g., to help maintain negative pressure achieved by a vacuum assisted closure device at a wound site), help fill dead space in the wound, help pull and/or direct edges of one or more portions and/or layers of the wound together as the wound heals, help form part of the new skin structure as the wound heals, help reduce wound size by bringing the edges of one or more portions of the wound together and to help encourage primary wound closure, and/or to perform any other suitable function.

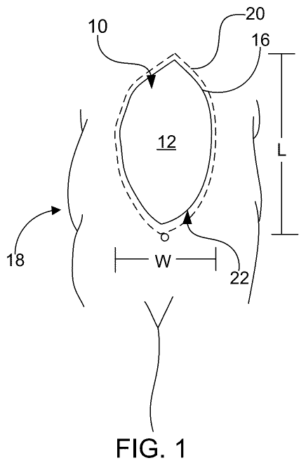

While the described biocompatible wound dressing can comprise any suitable component or characteristic that allows it function as intended, FIGS. 1-6 illustrate some embodiments in which the biocompatible wound dressing 10 comprises one or more biocompatible interfaces 12, fixation elements 14, and/or any other suitable component that allows the biocompatible interface to be inserted into a wound 16 of a patient 18 such that a portion (e.g., a perimeter 20) of the interface extends under, and is coupled to, a piece of tissue in the patient (e.g., under a perimeter of the skin 22 or other tissue surrounding the wound).

With respect to the biocompatible interface 12, the interface can comprise any suitable biocompatible material that is capable of: being inserted into a wound; acting as an occlusion dressing (e.g., for negative pressure wound therapy); filling a dead space in the wound; being at least one of absorbed and incorporated into the body; helping to form a matrix into which skin, blood vessels, fibroblasts, and/or epithelial cells can migrate and grow; helping to form skin over the wound; helping the wound to close; helping to pull one or more layers of a wound together; performing one or more of the roles of normal skin (e.g., keeping pathogens outside of the body, preventing desiccation of tissues below (or covered by) the biocompatible interface, and/or performing any other suitable function); being biodegradable; maintaining a three-dimensional structure within the wound for a long enough period of time to allow for ingrowth of blood vessels, fibroblasts, and/or epithelial cells; reducing the wound size and encouraging primary closure; and/or otherwise helping the wound to heal.

Some non-limiting examples of suitable biocompatible interfaces 12 include one or more materials comprising one or more dermal substitutes; pieces of cadaver skin (e.g., one or more pieces of cadaver dermis, reticular layers, and/or any other portion or portions of cadaver skin); pieces of ovine tissue and/or extracellular matrices of ovine forestomach; pieces of bovine tissue (e.g., one or more pieces of unilayer, bilayer, and/or any other suitable type of bovine tissue); pieces of bovine forestomach; pieces of porcine acellular dermal matrix; pieces of prolene; pieces of polypropylene; pieces of polytetrafluoroethylene (PTFE); pieces of E-polytetrafluoroethylene; pieces of Teflon™ material; pieces of Gortex™ material; pieces of polyurethane; pieces of surgical mesh; pieces of fully-resorbable surgical mesh; pieces of surgical mesh comprising poly (4-hydroxybutric acid) (P4HB); human adipose derived stem cells (hADSCs); stem cells; placental allografts; composite skin substitutes; cultured skin substitutes; dermo-epidermal skin substitutes; bi-layered skin substitutes (e.g., any suitable bi-layered skin substitute, including, without limitation, bi-layered skin substitutes formed from neonatal skin (e.g., male foreskin), keratinocytes, fibroblasts, and/or any other suitable material in a bovine type I collagen lattice and/or in any other suitable lattice); bovine type 1 collagens, with primary fibroblasts, keratinocytes, isolated human keratinocytes, fibroblast cells that are cultured in a collagen gel, and/or any other suitable material; cultured primary normal human epidermal keratinocytes that are air exposed on a collagen matrix that is seeded with primary normal human dermal fibroblasts; human organotypic skin explant cultures; pieces of reconstructed bi-layered human skin; human skin (e.g., allografts, autografts, xenografts, and/or any other suitable type of human skin and/or skin); types of acellular dermis; autologous keratinocytes and fibroblasts in a collagen-glycosaminoglycan-glycan matrix; collagen scaffolds; multi-layered stratified epithelia (e.g., from the differentiation of human primary fibroblasts and keratinocytes that are taken form a donor and placed in a bovine collagen scaffold and/or any other suitable type of multi-layered stratified epithelium); pieces of reconstructed human skin; pieces of cultured skin; double-layered skin substitutes (e.g., skin substitutes comprising an outer silicone layer and an inner layer formed with neonatal fibroblasts (e.g., from male foreskin and/or any other suitable source) and/or any other suitable skin substitutes having at least two layers); cryopreserved, split thickness allografts containing epidermis and/or partial dermis; pieces of human dermal matrix; pieces of human fibroblasts in a silicone sheet; skin substitutes comprising bovine collagen and/or chondroitin; skin substitutes comprising porcine collagen and/or nylon; pieces of porcine small intestinal submucosa; pieces of foam; types of spray-in and/or injectable foam; pieces of gauze; pieces of metal; pieces of plastic; polymers; ceramics; screens; synthetic materials; natural materials; bioabsorbable materials; non-bioabsorbable materials; pieces of tissue scaffolding; resilient materials; elastic materials; materials configured to have static shapes; and/or any other suitable biocompatible material that can function as all or part of the biocompatible interface. Indeed, in some embodiments, the biocompatible interface is not (or comprises one or more elements that are not) configured to be absorbed or permanently incorporated in the patient (e.g., the interface, or a portion thereof, is configured to be removed). In some embodiments, however, the biocompatible interface comprises a combination of materials that are biocompatible and/or bioabsorbable and/or that serve as a scaffolding or support to the soft tissue.

In some embodiments, for instance, the biocompatible interface 12 comprises one or more: pieces of cadaver skin (e.g., one or more pieces of cadaver dermis, reticular layers, and/or any other portions of cadaver skin); ovine tissue and/or extracellular matrices; pieces of ovine forestomach; pieces of bovine tissue (e.g., unilayer, bilayer, and/or any other suitable type of bovine tissue); pieces of porcine acellular dermal matrix; materials comprising prolene; materials comprising polypropylene; materials comprising polytetrafluoroethylene (PTFE); materials comprising E-polytetrafluoroethylene; materials comprising Teflon™ polymers; materials comprising Gortex™ polymers; materials comprising polyurethane; surgical meshes; fully-resorbable surgical meshes; pieces of surgical mesh comprising poly (4-hydroxybutric acid) (P4HB); human adipose derived stem cells (hADSCs); stem cells; and/or placental allografts. Indeed, in some embodiments, the biocompatible interface comprises cadaver skin, ovine forestomach, human adipose derived stem cells, and/or placental allograft.

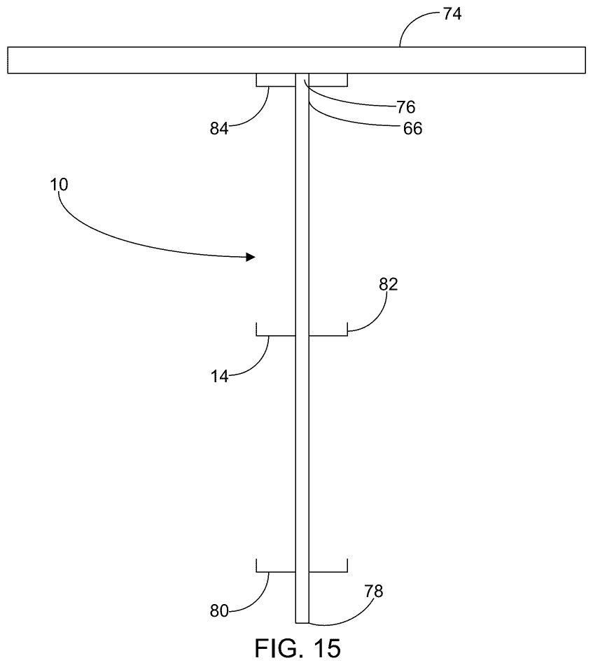

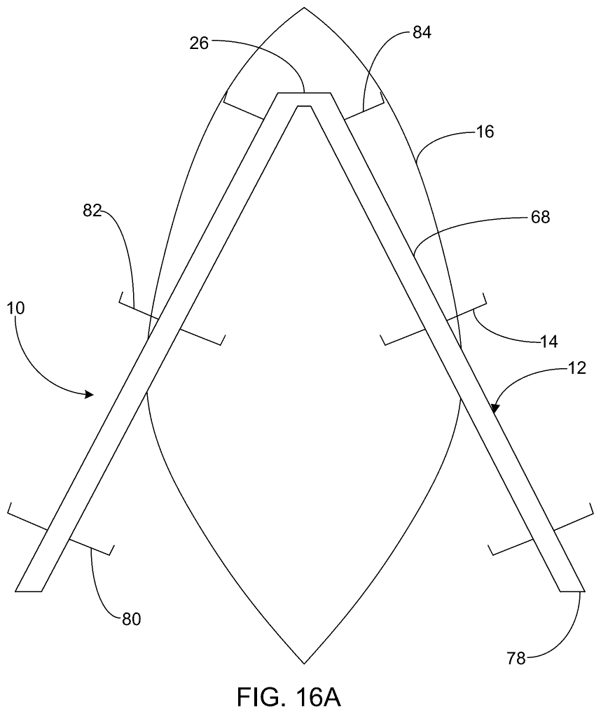

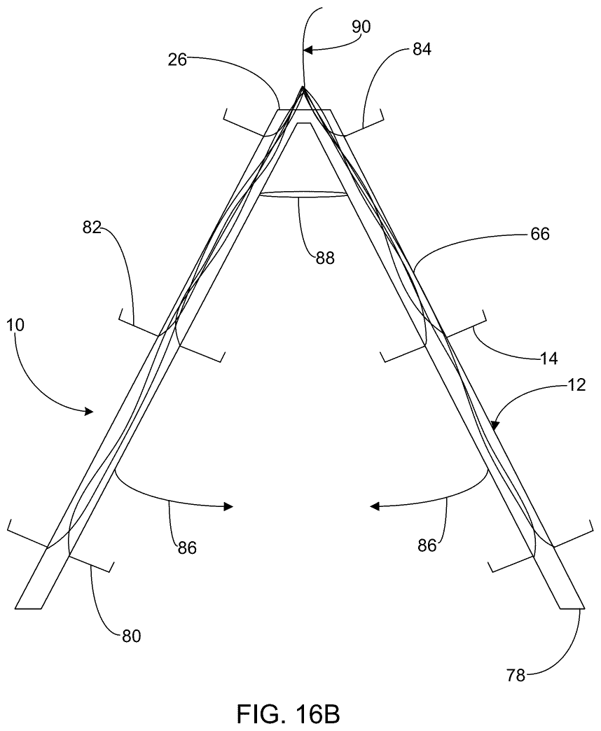

The biocompatible interface 12 can have any suitable shape that allows it to function as intended. Indeed, in some embodiments, the biocompatible interface comprises one or more: sheets of material (e.g., as illustrated in FIGS. 1-5A), posts (e.g., as illustrated by post 66 in FIG. 15), legs (e.g., as illustrated by legs 68 in FIG. 16), rods, shafts, objects having a ribbon shape, flexible materials, resilient materials, ropes, cables, linkages, objects having a V-shape, objects having a U-shape, elongated objects, bands, leaf shaped materials, and/or any other suitable shape. Indeed, in some embodiments, the biocompatible interface comprises one or more sheets of material or otherwise has a sheet-like shape.

Where the biocompatible interface 12 comprises a sheet of biocompatible material, the sheet of biocompatible material can be any suitable shape, including, without limitation, being circular, elliptical, triangular, square, rectangular, trapezoidal, polygonal, symmetrical, asymmetrical, regular, irregular, variable, substantially matching a shape of a corresponding wound (or a portion of the wound), and/or being any other suitable shape. By way of non-limiting illustration, FIG. 1 shows that, in some embodiments, the biocompatible interface 12 comprises a sheet of biocompatible material that is substantially elliptical in shape and/or that has a shape that somewhat resembles the perimeter of a football and/or of the wound 16.

Indeed, in some embodiments, the biocompatible interface 12 has a shape that is configured to substantially match that of the wound 16 it is being used to treat. For instance, in some embodiments, the wound is used as a stencil, guide, template, outline, perimeter, approximation, and/or model for forming the biological interface. In some such embodiments, once the shape of the wound is determined, the interface is cut, torn, 3D printed, customized to fit, trimmed, molded, and/or otherwise shaped or formed to substantially match the shape of the wound.

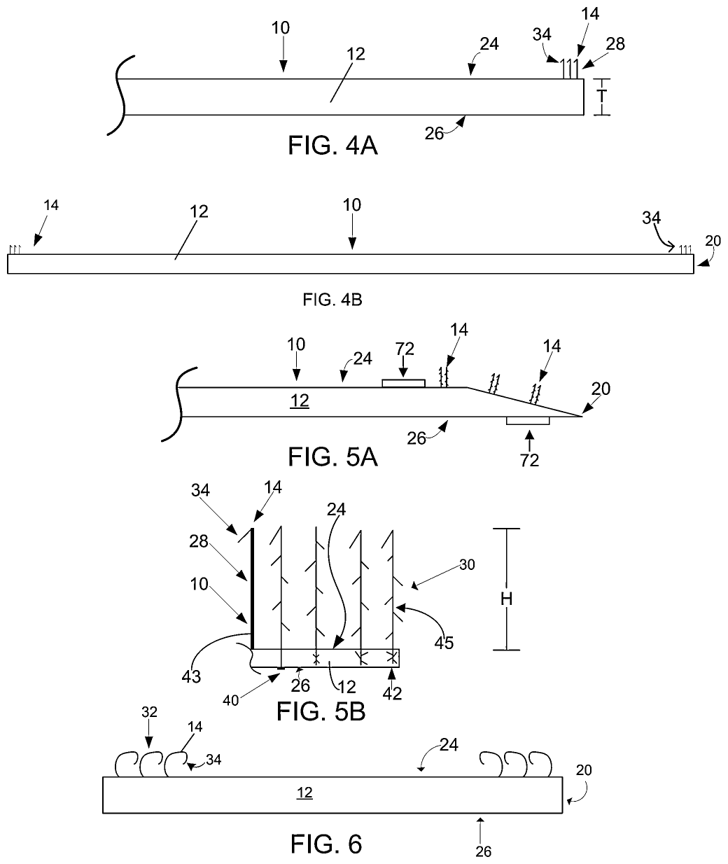

The biocompatible interface 12 can be any suitable size (e.g., length, width, depth, height, diameter, and any other suitable measurement) that allows it to function as described herein. For instance, the biocompatible interface can be any suitable thickness (e.g., having a suitable distance between a first surface 24 and an opposite second surface 26 of the interface where the interface comprises a sheet of material), including, without limitation, having a thickness (T, as illustrated in FIG. 4A) between 0.001 mm thick and 2 cm thick, or within any subrange thereof. Indeed, in some embodiments, the interface has a thickness that is between 1 mm and 1 cm. In some other embodiments, the interface has a thickness that is 1 cm thick±0.4 cm.

The biocompatible interface 12 can also have any suitable width and/or length (e.g., as shown by W and L, respectively, in FIG. 1) that allows it to function as intended. Indeed, in some embodiments, the interface has a width and/or length (e.g., where the interface comprises a sheet of one or more materials) that is between about 5 mm and about 1 m (or within any subrange thereof). Indeed, in some embodiments, the interface has a width and/or length that is between 2 cm and 50 cm. For instance, in some embodiments, the interface has a maximum width and/or maximum length that are each between about 5 cm and about 40 cm.

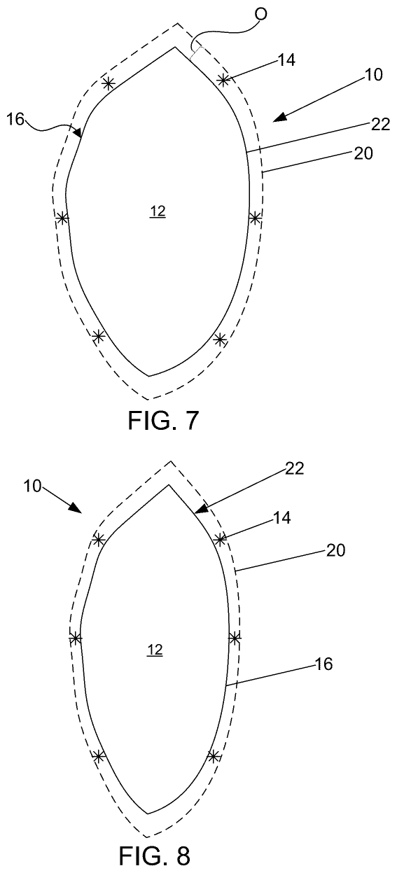

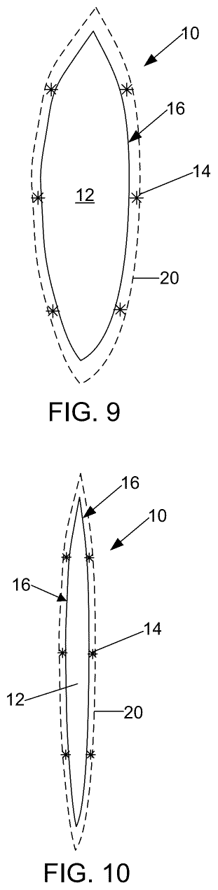

In some embodiments, the size (e.g., the length, width, depth, height, diameter, and any other suitable measurement) of the biocompatible interface 12 is based on the size of the wound 16 it is being used to treat. Indeed, in some embodiments in which the biocompatible interface comprises a sheet of material, the interface is smaller than the wound it is being used to treat such that the skin (or other tissue) around the wound needs to be stretched initially to couple with the interface. In some other embodiments, the biocompatible interface is roughly (or exactly) the same size as the wound it is being used to treat. In still other embodiments, the biocompatible interface is larger than the wound it is being used to treat such that one or more portions of the interface are configured to overlap with one or more pieces of the skin 22 (or other tissue) surrounding the wound.

Where one or more portions of the interface 12 (e.g., the perimeter 20) are configured to extend under a portion of the skin (and/or any other suitable tissue) surrounding the interface (e.g., when the skin (or other tissue) is at rest and/or when the skin (or other tissue) is stretched to cover a portion of the interface), any suitable amount of the interface can extend under (and/or overlap with) the skin (or other tissue). By way of non-limiting illustration, FIG. 7 shows that in some embodiments, one or more portions of the skin 22 surrounding the wound 16 overlap the interface 12 (or have an overlap O from one or more perimeters 20 of the interface 12). In some such embodiments, the interface is sized and shaped to have an overlap O with one or more portions of the skin (or other tissue) of between 0.1 mm and 15 cm (or within any subrange thereof). In some embodiments, the interface is configured to have an overlap of between 4 mm and 2 cm. In some other embodiments, the biological interface is sized to have an overlap O with one or more portions of the surrounding skin of between about 5 mm and about 1.5 cm.

With reference now to the fixation elements 14, the wound dressing 10 can have any suitable type of fixation elements that are capable of attaching the biocompatible interface 12 to skin 22 and/or any other suitable tissue that is at an edge of the wound 16 that is being treated. In this regard, some non-limiting examples of suitable fixation elements include one or more barbs, hooks, barbed shafts, hooked shafts, catches, clips, clamps, spikes, pins, sutures, clasps, grapnels, staples, anchors, suture anchors, metal anchors, adhesives (e.g., one or more bio-adhesives, fibrin sealants, fibrin glues, N-butyl 2-cyanoacrylate adhesives, 2-octyl cyanoacrylate, n-2-butyl-cyanoacrylate, gelatin formaldehyde/glutaraldehyde, and/or any other suitable type of adhesives), zip stitch sutures, zipper bandages, screws, absorbable screws, tacks, coiled tacks, uncoiled tacks, metal tacks, permanent acetal fasteners (e.g., PermaFix™-PF fastener material), titanium coil tacks (e.g., ProTack™-ST material), components resembling the hook and/or loop portion of a hook and loop fastener, fictional engagements, mechanical engagements, and/or any other suitable object that is capable of coupling the interface to the surrounding tissue. Indeed, in some embodiments, the fixation elements comprise one or more hooks, hook-like projections, adhesives, barbed shafts, and/or barbs. By way of non-limiting illustration, FIGS. 4A-6 show some embodiments in which one or more of the fixation elements 14 comprises a barbed shaft 28 (see FIG. 4A), a shaft having multiple projections 30 (see FIG. 5B), and a hook-shaped projection 32 (see FIG. 6).

In some embodiments in which the fixation elements 14 comprise one or more hooks, hook-like members, barbs, and/or barb-like members, the hooks, barbs, and/or projections can be oriented in any suitable manner, including, without limitation, opening towards (or having a free end that extends towards) a medial portion, a lateral portion, a perimeter edge, a proximal portion, a distal portion, a top end, a bottom end, the first surface 24, the second surface (or side) 26, and/or any other suitable portion of the biocompatible interface 12. In some embodiments, however, one or more (e.g., a significant number, a majority, and/or all) of the fixation elements are configured to open towards and/or have a free end that extends towards a center and/or medial portion of the interface. By way of non-limiting illustration, FIGS. 4A, 4B, 5B, and 6 show some embodiments, in which the fixation elements 14 are configured to open (or have a free end 34 that extends) towards a medial portion of the interface 12. While the orientation of the fixation elements can perform any suitable function, in some embodiments, by having the fixation elements open towards and/or have a free end or barb that extends towards a central and/or medial portion of the biocompatible interface, the fixation elements can act as springs, catches, fasteners, guides, adhesives, and/or are otherwise be configured to help catch tissue and/or to help direct such tissue towards a center and/or medial portion of the wound (e.g., to help the wound to close).

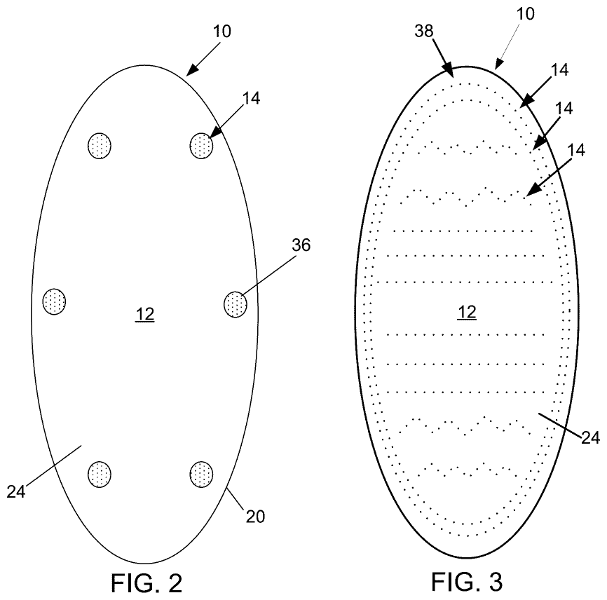

The fixation elements 14 can be disposed in any suitable location or locations that allow them to couple the biocompatible interface 12 to tissue (e.g., a subcutaneous layer of skin 22 surrounding the wound 16 and/or to any other suitable tissue). In some embodiments in which the biocompatible interface 12 comprises the first surface 24 (or a surface that is configured to act as an external surface of the interface during use) and the second surface 26 (or a surface that is configured to act as an internal surface during use), the fixation elements extend from and/or are disposed at: the first surface, the second surface, a perimeter between the first and second surface, an edge of the interface, a facet of the interface, a leg, a post, an anterior portion, a posterior portion, a lateral portion, a medial portion, a superior portion, an inferior portion, and/or any other suitable portion of the biocompatible interface. Indeed, in some embodiments, the interface comprises one or more fastener elements (e.g., hooks and/or adhesives) on its first surface and one or more fastener elements (e.g., adhesives and/or hooks) on its second surface. In accordance with some embodiments, however, FIGS. 4A-6 show that the fixation elements 14 are configured to extend from (and/or be disposed at) the first surface 24 of the biocompatible interface 12. Thus, in some such embodiments, when the interface is placed into a wound and the perimeter 20 of the interface is tucked under skin surrounding the wound, the fixation elements can catch, adhere to, or otherwise couple with subcutaneous tissue (and/or any other suitable portion) of the skin surrounding the wound to help fix or otherwise couple the interface to the skin.

Where the fixation elements 14 extend from (or are disposed at) the first surface 24 of the biocompatible interface 12, the fixations elements can be disposed in any suitable locations and/or patterns, including, without limitation, in one or more groups, rows, columns, lines, perimeters, central locations, lateral locations, superior locations, inferior locations, random locations, locations that are customized to the wound, and/or in any other suitable manner. By way of non-limiting illustration, FIG. 2 shows an embodiment in which the fixation elements 14 are disposed in one or more islands and/or other groupings 36. Additionally, FIG. 3 shows an embodiment in which the fixation elements 14 are disposed in one or more rows 38, adjacent to one or more perimeters 20 of the interface 12 (e.g., adjacent to a first lateral side, a second lateral side, a top, a bottom, and/or any other suitable portion of the interface 12.

The fixation elements 14 can be coupled to the biocompatible interface 12 in any suitable manner, including, without limitation, by being integrally formed with the interface; by being anchored within the interface; by being inserted into the interface; by being adhered to the interface; by having an enlarged back side (or end portion) that is configured to rest against the second side 26, an inside surface of the interface, and/or any other suitable portion of the interface; and/or in any other suitable manner. By way of non-limiting illustration, FIG. 5A shows an embodiment in which an adhesive 72 is disposed on the first 24 and/or second 26 surface. Additionally, FIG. 5B show some embodiments in which one or more fixation elements 14 have one or more enlarged ends 40 that are disposed at the second side 26 of the interface 12 to prevent the fixation elements from being pulled through the interface. Additionally, FIG. 5B shows an embodiment in which one or more of the fixation elements 14 comprise one or more anchors 42 that anchor the fixation elements to the interface 12.

Where the fixation elements 14 extend from the first 24 or any other suitable surface or portion of the biocompatible interface 12, the fixation elements can extend to any suitable length or height H (e.g., as shown in FIG. 5B) that allows them to couple the biocompatible interface to adjacent tissue (e.g., subcutaneous tissue of skin 22 surrounding the wound 16 and/or any other suitable tissue). In some embodiments, the fixation elements extend from the first surface (or any other suitable part) of the biocompatible interface by a height H (or distance) of between about 0.1 mm and about 2.5 cm (or within any subrange thereof). For instance, some embodiments of the fixation elements extend from the interface to a height H or distance that is between 0.2 mm and 0.8 cm. In some embodiments, the fixation elements extend from the biocompatible interface to a height of 1 cm±5 mm.

The fixation elements 14 can comprise any suitable material or materials that allow the elements to couple the biocompatible interface 12 to one or more tissues of a user (e.g., skin, subcutaneous tissue, muscle, facia, fatty tissue, and/or any other suitable tissues). Some non-limiting examples of suitable materials include one or more synthetic polymer materials (including, without limitation, polydioxanone, poly(p-dioxanone), polyglycolic acid, poly(glycolic) acid, polyglyconate, polylactic acid, poly(l-lactic acid), poly-Llactide (PLIA), poly(lactic acid-co-glycolic acid), polylactide acid, polyglactin 910, Vicryl™ material, Monocryl® suture material, copolymers of glycolide and e-caprolactone, poly(lactide-co-glycolide), polyester coated with polybutylate, Ethibond™ suture material, synthetic polymers of polyglycolide (e.g., pure polyglycolide and/or any other suitable polyglycolide), polyglactin fast, polyglactin, poliglecaprone, polytrimethylene carbonate, and/or any other suitable synthetic polymer materials), natural fibers (including, without limitation, purified catgut, collagen, sheep intestines, cow intestines, plain gut, and/or any other suitable natural fibers), catgut chromic, poliglecaprone, polyglactine irradiated, metal (e.g., titanium, titanium alloy, stainless steel, cobalt-chromium alloy, platinum, palladium, and/or any other suitable metal or metals), wire, nylon, silk, polypropylene, prolene, polyester, PVDF, PTFE, UHMWPE, biodegradable/bioabsorbable synthetic polymer compounds, and/or any other suitable biocompatible material. By way of non-limiting example, in some embodiments, the fixation elements comprise one or more biocompatible and/or bioabsorbable materials. In some embodiments, the fixation elements comprise one or more mono-filamental, multi-filamental, braided, woven, twisted, and/or non-braided structures that are configured to catch or otherwise couple with tissue surrounding the biocompatible interface. In some such embodiments, the fixation elements comprise one or more absorbable materials (e.g., poly(lactide-co-glycolide), poly(p-dioxanone), one or more copolymers of glycolide and e-caprolactone, and/or any other suitable biocompatible and absorbable material that can be used to form the fixation elements). In some embodiments, however, the fixation elements comprise one or more non-absorbable materials (e.g., nylon, prolene, braided polyester coated with polybutylate, wire, and/or any other suitable biocompatible material that is not intended to be absorbed in a patient).

Indeed, in some embodiments, one or more of the fixation elements 14 are not configured to be absorbed and/or incorporated into the user's body. In some such embodiments, the fixation elements can stay in place permanently, can be removed when desired, and/or can stay in place until the biocompatible interface 12 is absorbed and/or incorporated into the body of the patient being treated (e.g., where the biocompatible interface is configured to be absorbed and/or incorporated into the patient).

In still some other embodiments, however, one or more of the fixation elements 14 are configured to be absorbed and/or incorporated into the user's body. In such embodiments, the fixation elements are configured to be absorbed and/or incorporated into the body at roughly the same time as, before, and/or after the biocompatible interface is absorbed and/or incorporated into the user's body (e.g., where the interface is configured to be absorbed and/or incorporated into the body). Indeed, in some embodiments, over a period of time (e.g., between 1 day and 24 weeks, or any subrange thereof), the fixation elements are configured to be absorbed and/or incorporated into the body of the user and/or the biocompatible interface is absorbed and/or incorporated into the user's body. Indeed, in some embodiments, the biocompatible interface and the fixation elements are configured to be incorporated into the patient's body.

In addition to the aforementioned characteristics, the fixation elements 14 can have any other suitable component or characteristic. Indeed, in some embodiments, one or more of the fixation elements comprise one or more monofilament portions (e.g., shafts, barbs, and/or any other suitable portion), multifilament portions (e.g., shafts, barbs, and/or any other suitable portion), braided portions (e.g., shafts, barbs, and/or any other suitable portion), twisted portions (e.g., twisted shafts, barbs, and/or any other suitable portion or portions), non-braided portions, barbed structures, hooked structures, catches, coatings (e.g., silicon coatings, wax coatings, PTFE coatings, polycaprolactone coatings, calcium stearate coatings, antimicrobial coatings, and/or any other suitable type of coating or coatings), impregnations (e.g., antimicrobials, hormones, and/or any other suitable type of impregnable material), and/or have any other suitable characteristic or characteristics. By way of non-limiting illustration, FIG. 5B shows that, in some embodiments, one or more fixation elements 14 comprise one or more braided elements 43 and/or a multi-barbed elements 45. In some embodiments, however, the fixation elements comprise a monofilament biocompatible material that is configured to be incorporated into soft tissue.

In addition to the aforementioned components, the described wound dressing 10 can comprise any other suitable component. Some examples of such components include, but are not limited to, one or more: antimicrobial agents (e.g., one or more types of antibiotics, colloidal silver, placental allograft, antiseptics, and/or any other suitable antimicrobial), types of petroleum jelly, types of ointments, types of growth hormones, and/or any other suitable material or materials.

As another example of a suitable modification, some embodiments of the biocompatible interface 12 comprise one or tapered, textured, and/or feathered edges (e.g., to help speed integration and/or absorption of the interface into the user's body, to increase comfort of the interface, to increase ease of use, and/or for any other suitable purpose). By way of non-limiting illustration, FIG. 5A shows that in some embodiments, the perimeter 20 of the biocompatible interface 12 is tapered.

As another example of a suitable modification, some embodiments of the biocompatible interface 12 are configured to be placed horizontally, vertically, diagonally, and/or in any other suitable manner in or with respect to a wound 16. Indeed, the described wound dressing 10 can be configured to be used in any suitable manner that allows the dressing to help close a wound and/or to reduce dead space underlying skin. By way of non-limiting illustration, FIG. 15 shows an embodiment in which the wound dressing 10 comprises one or more posts 66 having one or more fixation elements 14 attached thereto, wherein the wound dressing is disposed vertically within a wound (e.g., to be substantially perpendicular to a skin surface 74). Moreover, FIG. 16 shows an embodiment in which the dressing 10 comprises a biocompatible interface 12 and one or more fixation elements 14, wherein one or more of such wound dressings are configured to be inserted into a wound, at any suitable orientation (e.g., horizontally; vertically, with a top end 76 disposed at or adjacent to an opening of the wound and one or more bottom ends 78 disposed deeper within the wound), or in any other suitable manner. Indeed, in some embodiments, the wound dressing 10 of FIG. 16 is configured to be disposed vertically within the wound 16.

Where the wound dressing 10 does not consist of a sheet of a biocompatible interface 12 with fasteners where the sheet is configured to lay horizontally across a wound (e.g., to run substantially parallel with the skin surrounding the wound), but instead comprises one or more rods; posts; legs; shafts; ribbons; objects having a ribbon shape; resilient items; elastic items; flexible materials; ropes; cables; linkages; objects having a V-shape; objects having a U-shape; elongated objects; bands; leaf shaped materials; and/or any other suitable shaped interfaces that are configured to be disposed vertically, horizontally along a length of the wound, diagonally in the wound, and/or in any other suitable manner, the wound dressing can comprise any suitable component or characteristic. Indeed, in some embodiments, the biocompatible interface and/or the fixation elements comprise any of the corresponding materials described herein. Indeed, in some embodiments, the biocompatible interfaces 12 and/or the fixation elements 14 of FIGS. 15 and 16 comprise one or more biocompatible and absorbable materials (as described herein) that are configured to be absorbed by and/or incorporated into the patient's body. In some embodiments, however, the biocompatible interface and/or the fixation elements comprise one or more materials (e.g., one or more plastics, polymers, metals, ceramics, and/or any other suitable non-absorbable materials) that are not configured to be absorbed by and/or incorporated into the patient's body. Indeed, in some embodiments, the biocompatible interface and/or fixation elements are configured to be extracted from the wound (e.g., as the wound heals).

Where the wound dressing 10 does not consist of a sheet of material comprising one or more fixation elements 14 (e.g., as shown in FIGS. 15 and 16), the fixation elements can be disposed (as described above) at any suitable location with respect to biocompatible interface 12 that allows the wound dressing to function as described herein. Indeed, in some embodiments, the wound dressing comprises fixation elements that are configured to couple the dressing to different types and/or pieces of tissue. Indeed, in some embodiments, the wound dressing comprises one or more fixation elements that are configured to couple the interface to one or more pieces of muscle, muscle facia, facia, fatty tissue, subcutaneous tissue, skin, organ, and/or any other suitable tissue. Accordingly, the fixation elements are, in some embodiments, disposed in different locations that allow them to couple to different pieces of tissue. By way of non-limiting illustration, FIGS. 15-16 show some embodiments in which the wound dressing 10 comprises one or more fixation elements 14 that are configured to couple with: muscle and/or facia (see elements 80), subcutaneous fat (see elements 82), dermis (see elements 84), and/or any other suitable tissue. Thus, some embodiments of the wound dressing allow for layered fastening of tissue to reduce dead space underlying skin.

Although some embodiments of the biocompatible interface 12 are rigid (or comprise rigid portions), in some embodiments, all or a portion of the biocompatible interface are malleable, resilient, flexible, biased, configured to be incrementally tightened or adjusted, and/or otherwise made to have their configuration change. Indeed, in some embodiments in which the biocompatible interface has two or more legs, posts, shafts, ribbons, and/or other elongated members, one or more of the members are configured to move (or to be biased to help move) tissue together to help heal a wound. By way of non-limiting illustration, FIG. 16B shows that in some embodiments, the legs 68 are biased together (e.g., as illustrated by arrows 86) so as to help pull muscle and/or muscle facia together, to pull opposing pieces of subcutaneous facia together, to pull pieces of dermis together, and/or to perform any other suitable purpose.

Where the wound dressing 10 comprises one or more elongated members and/or other objects that are biased or configured to move as the healing process takes place, such members and/or other objects can be moved or biased in any suitable manner, including, without limitation, through the use of one or more springs, elastics, biasing materials, resilient materials, cords, clamps, adjustable clamping mechanisms, threaded engagements, mechanical engagements, and/or in any other suitable manner. By way of non-limiting illustration, FIG. 16B shows an embodiment in which two legs 68 of the wound dressing 10 are biased together through the use of one or more elastics 88.

While some embodiments of the biocompatible interface 12 are configured to be absorbed by or to be integrated into a patient's body, some embodiments of the biocompatible interface (as previously mentioned) are configured to be removed from the patient. In such embodiments, the interface can be removed from the patient in any suitable manner. Indeed, in some embodiments, a non-absorbable interface (or interface that less absorbable than the fixation elements 14) can be removed when all or a portion of the fixation elements have been absorbed in the patient's body. Additionally, in some embodiments, one or more of the fixation elements are configured to be retracted into and/or from the biocompatible interface (e.g., via one or more mechanical mechanisms, winches, push-button extraction mechanisms, ratchets, and/or via any other suitable mechanism. By way of non-limiting illustration, FIG. 16B shows that, in accordance with some embodiments, one or more of the fixation elements 14 comprise and/or are coupled to a wire, string, filament, or other coupler that extends from and/or through the biocompatible interface 12 such that a release 90 can be pulled (or otherwise actuated) to pull the fixation elements into the interface and to release the interface from the patients (e.g., such that the interface can be extracted without harming the patient.

As still another example of a suitable modification, some embodiments of the described wound dressing 10 are used with one or more sensors and/or processors. In this regard, the wound dressing can be used with any suitable type of sensor or sensors, including, without limitation, one or more proximity sensors, moisture sensors, temperature sensors, movement sensors, pH sensors, stress sensors, tension sensors, pressure sensors, humidity sensors, contact sensors, GPS sensors, and/or any other suitable sensors. In such embodiments, the sensors (in conjunction with one or more processors) can perform any suitable function, including, without limitation, identifying if two or more portions (e.g., sides) of a wound 16 are coming together, staying static, moving apart, and/or moving in any other suitable manner; determining if the skin 22 surrounding the biocompatible interface 12 is fixed (or moving) with respect to the interface; identifying whether the negative pressure device should increase or decrease a negative pressure of the wound; determining if there are fluids that need to be removed from the wound; identifying if there are one or more signs of infection around the wound; identifying excessive tension and/or pressure in connection with the wound; and/or performing any other suitable function.

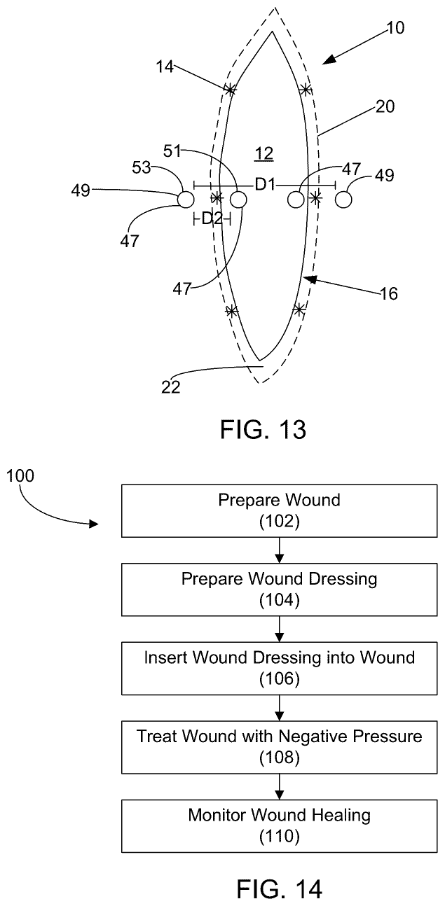

By way of non-limiting illustration, FIG. 13 shows a representative embodiment, in which the wound dressing 10 and/or surrounding skin 22 comprise one or more sensors 47 that are configured to determine if a wound 16 is closing and/or healing properly (e.g., if the sensors are properly aligned and moving closer to each other over time, for instance to see if the distance D1 between the first set of sensors 49 is reducing as expected). Additionally, FIG. 13 shows an embodiment in which a first sensor 51 is disposed at the biocompatible interface 12 and a second sensor 53 is disposed on a nearby piece of skin 22 to determine if a distance D2 between the first sensor 51 and the second sensor 53 is increasing, decreasing, remaining static, aligned, misaligned, and/or otherwise changing.

As another example of a suitable modification, the described systems and methods can be used in a manner that is similar to an orthopedic distraction device (e.g., to help maintain a space and/or tension and/or to help in cellular proliferation).

As yet another example of a suitable modification, in some embodiments, the biocompatible interface is configured to be injected and/or sprayed into a patient. In such embodiments, the biocompatible interface can have any suitable component or characteristic. For instance, in some embodiments, the biocompatible interface comprises a sprayable or injectable embodiment of any of the materials described herein for use with the interface. Indeed, in some instance, the biocompatible interface comprises a sprayable foam.

As an additional example of a suitable characteristic of the biocompatible interface 12 (where the interface is configured to be injected or sprayed into a patient), some embodiments of the interface are configured to be moldable for a period of time (e.g., to properly fill a wound 16, to receive and retain one or more fixation elements 14, and/or for any other suitable purposes) and to then harden, congeal, become rubbery, become viscoelastic, cure, or otherwise harden for use. Thus, in some embodiments, an injectable or sprayable version of the interface can be coupled to the patient via one or more fixation elements that are coupled to and/or attached in the interface.

In addition to the aforementioned features, the described wound dressing 10 can have any other suitable feature that allows it to operate as intended. Indeed, in some embodiments, described wound dressing can: help wounds to heal faster and/or better than may otherwise happen, reduce chances of infection, increase comfort for a user, preserve mobility for a user, encourage primary healing, pull various pieces of tissue together, and/or perform any other suitable function.

The described wound dressing 10 can also be made in any suitable manner. In this regard, some non-limiting examples of methods for making the described wound dressing include cutting, sewing, stitching, punching, fitting, culturing, welding, melting, 3D printing, molding, customizing the wound dressing to wound size and/or dimension, injecting or spraying the biocompatible interface 12 into a patient, curing the interface, coupling one or more fixation elements to the biocompatible interface, biasing one or more portions of the interface, and/or otherwise forming and assembling the various components to form the wound dressing.

The described wound dressing 10 can also be used in any suitable manner. In this regard, FIG. 14 includes an example of a suitable method 100 for using the wound dressing, noting that such method can be rearranged, modified, have one or more elements added to it, have one or more elements be omitted from it, have one or more portions be performed in series, have one or more portions be performed in parallel, have one or more portions be repeated, have one or more portions be substituted with another portion, and/or otherwise be modified in any suitable manner. In any case, FIG. 14 shows that, in some embodiments, the method 100 includes (as shown at box 102) preparing a wound (e.g., by forming, debriding, recutting, cleaning, preparing a wound bed, disinfecting, washing, scrubbing, and/or otherwise treating the wound).

Moreover, box 104 shows that some embodiments of the method 100 continue as the wound dressing is prepared (e.g., the wound dressing: is formed, removed from a sterile container, cut to the proper size and/or shaped for usage, wetted with one or more desired fluids (e.g., one or more types of antimicrobial, growth hormone, water, saline, antiseptic solution, and/or any other suitable fluid), treated by having one or more fixation elements 14 be coupled to the biocompatible interface 12, and/or is otherwise prepared for being inserted into the wound).

Box 106 shows that, in some embodiments, the method 100 continues as one or more of the perimeters 20 of the of the biocompatible interface 12 are inserted below one or more portions of the skin 22 surrounding the biocompatible interface, with one or more fixation elements 14 being coupled to the skin (e.g., at a subcutaneous layer of the skin) and/or to any other suitable tissue.

Box 108 shows that, in some embodiments, the method 100 continues as one more negative pressure wound therapy devices are optionally used on the wound (e.g., to draw the wound closed, to remove exudate from the wound, to reduce pressure around the wound, reduce wound size, encourage primary healing, and/or for any other suitable purpose. While the negative pressure wound therapy device can be used in any suitable manner, in some embodiments, a sealed dressing is applied to the wound (with or without a breathable foam or packing), the sealed dressing is attached to tubing that, in turn, is attached to a pump (or vacuum), and the pump is selectively actuated. In some embodiments, in addition to or in place of use of a pump to help close the wound, one more zip stitch sutures, zipper bandages, clamps, forceps, and/or any other suitable mechanical closure devices are used to help close the wound.

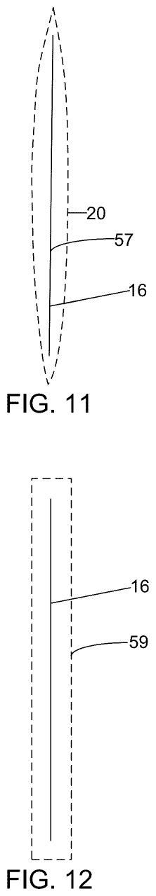

Box 110 shows that, in some embodiments, as the method 100 continues, one or more practitioners can monitor the wound's healing process (e.g., visually, by reviewing sensor readings, by measuring or checking measurements of the biocompatible interface (e.g., by checking how close the legs 68 are to each other), and/or in any other suitable manner). Additionally, the wound can otherwise be treated as part of the method. Thus, FIG. 11 shows that in some embodiments as the wound forms, a scar 57 forms. Moreover, FIG. 12 shows that, in some embodiments, as the wound heals, one or more bandages 59 are placed over the healing wound (e.g., to help it stay closed until it is properly healed). In short, in some embodiments, the described wound dressing 10 can be used to treat a wound in any suitable manner.

Thus, as discussed herein, the described systems and methods relate to the treatment of wounds. In particular, the described systems and methods relate to a biocompatible wound dressing that can include any suitable component. In some cases, it includes one or more biocompatible interfaces and one or more fixation elements (e.g., barbs, hooks, adhesives, catches, and/or any other suitable fixation elements) that are each configured to be absorbed and/or incorporated into a user's body after prolonged use in the user's body. In some cases, the interface has a first surface and a second surface that is disposed substantially opposite to the first surface, with a peripheral edge that extends around an outer perimeter of the biocompatible interface. In some cases, the fixation elements extend from (and/or are disposed at) the first surface of the interface, adjacent to the peripheral edge. In some cases, the fixation elements also have a hook-like shape that is configured to catch or otherwise couple the interface to subcutaneous tissue on an undersurface of skin when the peripheral edge and the fixation elements are inserted below the subcutaneous tissue. In some cases, the wound dressing is used with a negative pressure wound therapy device and/or any other suitable mechanical closure device.

### Representative Operating Environment

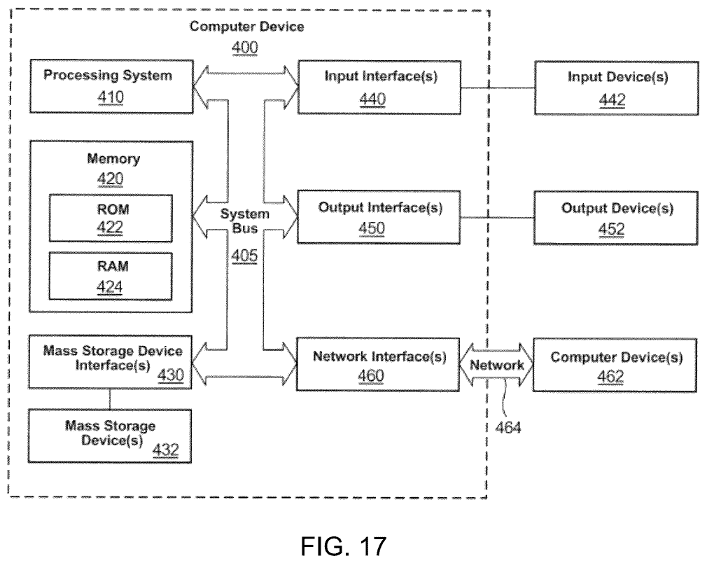

As mentioned previously, some embodiments of the described wound dressing 10 (e.g., embodiments that comprise one or more sensors 47) comprise (or are used with) one or more processors. In this regard, the described wound dressing 10, mechanical closure device, and/or negative pressure wound therapy device can be used with, or in, any suitable operating environment and/or software. In this regard, FIG. 17 and the corresponding discussion are intended to provide a general description of a suitable operating environment in accordance with some embodiments of the described systems and methods. As will be further discussed below, some embodiments embrace the use of one or more processing (including, without limitation, micro-processing) units in a variety of customizable enterprise configurations, including in a networked configuration, which may also include any suitable cloud-based service, such as a platform as a service or software as a service.

Some embodiments of the described systems and methods embrace one or more computer readable media, wherein each medium may be configured to include or includes thereon data or computer executable instructions for manipulating data. In accordance with some embodiments, the computer executable instructions include data structures, objects, programs, routines, and/or other program modules that can be accessed by one or more processors, such as one associated with a general-purpose processing unit capable of performing various different functions or one associated with a special-purpose processing unit capable of performing a limited number of functions (e.g., the processor 44). In this regard, in some embodiments, the processing unit comprises a specialized processing unit 44 that is configured for use with the described wound dressing 10.

Computer executable instructions cause one or more processors of the enterprise to perform a particular function or group of functions and are examples of program code means for implementing steps for methods of processing. Furthermore, a particular sequence of the executable instructions provides an example of corresponding acts that may be used to implement such steps.

Examples of computer readable media (including non-transitory computer readable media) include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing unit.

With reference to FIG. 17, a representative system includes computer device 400 (e.g., one or more processors), which may be a general-purpose or special-purpose computer (or processing unit). For example, computer device 400 may be one or more processors, personal computers, notebook computers, PDAs or other hand-held devices, workstations, minicomputers, mainframes, supercomputers, multi-processor systems, network computers, processor-based consumer devices, cellular phones, tablet computers, smart phones, feature phones, smart appliances or devices, control systems, artificial intelligence, machine learning devices, neural networks, negative pressure therapy devices, mechanical closure devices, and/or the like.

Computer device 400 includes system bus 405, which may be configured to connect various components thereof and enables data to be exchanged between two or more components. System bus 405 may include one of a variety of bus structures including a memory bus or memory controller, a peripheral bus, or a local bus that uses any of a variety of bus architectures. Typical components connected by system bus 405 include processing system 410 and memory 420. Other components may include one or more mass storage device interfaces 430, input interfaces 440, output interfaces 450, and/or network interfaces 460, each of which will be discussed below.

Processing system 410 includes one or more processors, such as a central processor and optionally one or more other processors designed to perform a particular function or task. It is typically processing system 410 that executes the instructions provided on computer readable media, such as on the memory 420, a magnetic hard disk, a removable magnetic disk, a magnetic cassette, an optical disk, or from a communication connection, which may also be viewed as a computer readable medium.

Memory 420 includes one or more computer readable media (including, without limitation, non-transitory computer readable media) that may be configured to include or includes thereon data or instructions for manipulating data, and may be accessed by processing system 410 through system bus 405. Memory 420 may include, for example, ROM 422, used to permanently store information, and/or RAM 424, used to temporarily store information. ROM 422 may include a basic input/output system (“BIOS”) having one or more routines that are used to establish communication, such as during start-up of computer device 400. RAM 424 may include one or more program modules, such as one or more operating systems, application programs, and/or program data.

One or more mass storage device interfaces 430 may be used to connect one or more mass storage devices 432 to the system bus 405. The mass storage devices 432 may be incorporated into or may be peripheral to the computer device 400 and allow the computer device 400 to retain large amounts of data. Optionally, one or more of the mass storage devices 432 may be removable from computer device 400. Examples of mass storage devices include hard disk drives, magnetic disk drives, tape drives, solid state mass storage, and optical disk drives.

Examples of solid-state mass storage include flash cards and memory sticks. A mass storage device 432 may read from and/or write to a magnetic hard disk, a removable magnetic disk, a magnetic cassette, an optical disk, or another computer readable medium. Mass storage devices 432 and their corresponding computer readable media provide nonvolatile storage of data and/or executable instructions that may include one or more program modules, such as an operating system, one or more application programs, other program modules, or program data. Such executable instructions are examples of program code means for implementing steps for methods disclosed herein.

One or more input interfaces 440 may be employed to enable a user to enter data (e.g., initial information) and/or instructions to computer device 400 through one or more corresponding input devices 442. Examples of such input devices include a keyboard and/or alternate input devices, such as one or more switches, buttons, dials, sensors (e.g., temperature sensors, G-force sensors, RPM sensors, color sensors, heart rate sensors, blood pressure sensors, conductivity sensors, sweat sensors, and/or any other suitable type of sensors, including, without limitation, those discussed elsewhere herein), digital cameras, pin pads, touch screens, mice, trackballs, light pens, styluses, or other pointing devices, microphones, joysticks, game pads, scanners, camcorders, and/or other input devices. Similarly, examples of input interfaces 440 that may be used to connect the input devices 442 to the system bus 405 include a serial port, a parallel port, a game port, a universal serial bus (“USB”), a firewire (IEEE 1394), a wireless receiver, a video adapter, an audio adapter, a parallel port, a wireless transmitter, or another interface.

One or more output interfaces 450 may be employed to connect one or more corresponding output devices 452 to system bus 405. Examples of output devices include a monitor or display screen, a speaker, a wireless transmitter, a printer, and the like. A particular output device 452 may be integrated with or peripheral to computer device 400. Examples of output interfaces include a video adapter, an audio adapter, a parallel port, and the like.

One or more network interfaces 460 enable computer device 400 to exchange information with one or more local or remote computer devices, illustrated as computer devices 462, via a network 464 that may include one or more hardwired and/or wireless links. Examples of the network interfaces include a network adapter for connection to a local area network (“LAN”) or a modem, BLUETOOTH™, Wi-Fi, a cellular connection, a wireless link, or another adapter for connection to a wide area network (“WAN”), such as the Internet. The network interface 460 may be incorporated with or be peripheral to computer device 400.

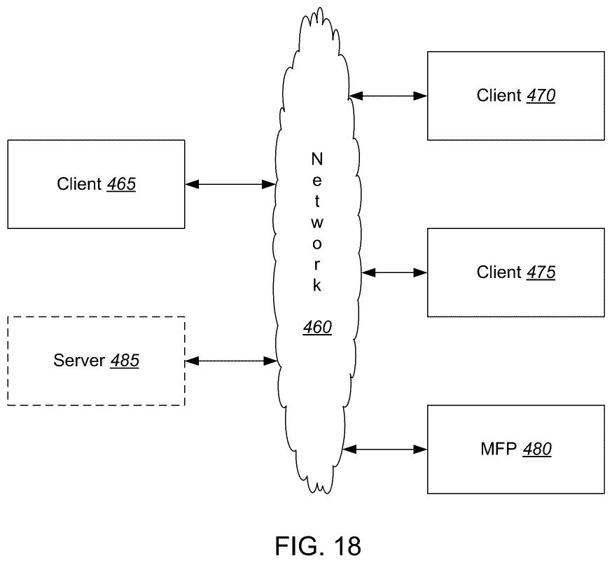

In a networked system, accessible program modules or portions thereof may be stored in a remote memory storage device. Furthermore, in a networked system computer device 400 may participate in a distributed computing environment, where functions or tasks are performed by a plurality networked computer devices. While those skilled in the art will appreciate that the described systems and methods may be practiced in networked computing environments with many types of computer system configurations, FIG. 18 represents an embodiment of a portion of the described systems (e.g., the wound dressing 10 with one or more sensors 47) in a networked environment that includes clients (465, 470, 475, etc.) connected to a server 485 via a network 460. While FIG. 18 illustrates an embodiment that includes 3 clients (e.g., the described wound dressing with one or more sensors and/or one or more negative pressure wound therapy devices) connected to the network, alternative embodiments include at least one client connected to a network or many clients connected to a network. Moreover, embodiments in accordance with the described systems and methods also include a multitude of clients throughout the world connected to a network, where the network is a wide area network, such as the Internet. Accordingly, in some embodiments, the described systems and methods can allow for remote: monitoring, modification, control, training, communication, observation, adjustment, troubleshooting, data collecting, system optimization, user interaction, and/or other controlling of the described wound dressing 10, one or more associated sensors 47, and/or one or more associated negative pressure therapy devices from one or more places throughout the world.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, implementations, cases, examples, and illustrations set forth herein can be combined in absolutely any suitable manner with any other components or characteristics from one or more other embodiments, implementations, cases, examples, and illustrations described herein.

In addition, as the terms on, disposed on, attached to, connected to, coupled to, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., front back, on top of, below, above, top, bottom, side, up, down, under, over, upper, lower, lateral, right-side, left-side, base, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

What is claimed is:

1. A biocompatible wound dressing, comprising:
a biocompatible interface; and
multiple fixation elements that are disposed on the biocompatible interface,
wherein the fixation elements are configured to couple the biocompatible interface to a subcutaneous tissue on an undersurface of skin when the fixation elements are inserted below the subcutaneous tissue.

2. The biocompatible wound dressing of claim 1, wherein the biocompatible interface is configured to be at least one of: (i) absorbed in a patient's body and (ii) incorporated into the patient's body.

3. The biocompatible wound dressing of claim 1, wherein the biocompatible interface comprises a sheet of absorbable material having a first surface and a second surface that is substantially opposite to the first surface, and wherein the fixation elements are disposed at the first surface of the biocompatible interface.

4. The biocompatible wound dressing of claim 1, wherein the biocompatible interface is configured to extend vertically into a wound, and wherein the fixation elements are disposed at different locations along a length of the biocompatible interface to allow the fixation elements to couple with multiple different types of tissues that are disposed at differing depts in the wound.

5. The biocompatible wound dressing of claim 4, wherein the fixation elements extend from the biocompatible interface from at least three different locations along the length of the biocompatible interface.

6. The biocompatible interface of claim 1, wherein the fixation elements are configured to be retracted into the biocompatible interface.

7. The biocompatible wound dressing of claim 1, wherein the biocompatible interface comprises two elongated elements that are biased together to help pull tissue together when the wound dressing is disposed in a wound.

8. The biocompatible wound dressing of claim 3, wherein the fixation elements only extend from first surface, and wherein the fixation elements are configured to be at least one of: (i) absorbed into a patient's body and (ii) incorporated into the patient's body.

9. A biocompatible wound dressing, comprising:
a biocompatible interface comprising a sheet of biocompatible material having:
a first surface;
a second surface that is disposed substantially opposite to the first surface; and
a peripheral edge that extends around an outer perimeter of the biocompatible interface; and
multiple fixation elements that are disposed at the first surface, adjacent to the peripheral edge, of the biocompatible interface, wherein the fixation elements are configured to couple the biocompatible interface to a subcutaneous tissue on an undersurface of skin when the peripheral edge and when the fixation elements are inserted below the subcutaneous tissue.

10. The biocompatible wound dressing of claim 9, wherein the biocompatible interface comprises at least one of: (i) cadaver tissue and (ii) ovine forestomach.

11. The biocompatible wound dressing of claim 9, wherein the fixation elements each have a hook shape and extend from the first surface of the biocompatible interface by less than 1 cm.

12. The biocompatible wound dressing of claim 9, wherein the biocompatible interface comprises a first lateral edge and a second lateral edge, with the second lateral edge being disposed substantially opposite to the first lateral edge, and wherein the fixation elements are disposed at both the first lateral edge and the second lateral edge of the biocompatible interface.

13. The biocompatible wound dressing of claim 9, wherein biocompatible interface is configured to at least one of: (i) absorb into a tissue space and (ii) incorporate into the tissue space after prolonged use of the biocompatible wound dressing in the tissue space.

14. The biocompatible wound dressing of claim 9, wherein the fixation elements comprise an adhesive.

15. A biocompatible wound dressing, comprising:
a biocompatible interface that is configured to at least one of: (i) absorb and (ii) incorporate into a user's body after prolonged use in the users' body, the biocompatible interface comprising a sheet of material having:
a first surface;
a second surface that is disposed substantially opposite to the first surface; and
a peripheral edge that extends around an outer perimeter of the biocompatible interface; and
multiple fixation elements that are configured to at least one of: (a) absorb and (b) incorporate into the user's body after prolonged use in the user's body,
wherein the fixation elements extend from the first surface, adjacent to the peripheral edge of the biocompatible interface, and
wherein each of the fixation elements comprises at least one of: (i) a hook shape and (ii) a barb that is configured to catch and thereby couple the biocompatible interface to a subcutaneous tissue on an undersurface of skin when the peripheral edge and fixation elements are inserted below the subcutaneous tissue.

16. The biocompatible wound dressing of claim 15, wherein the fixation elements have a catch portion that opens towards a medial portion of the biocompatible interface.

17. The biocompatible wound dressing of claim 15, wherein the peripheral edge is at least one of tapered and feathered.

18. A method for dressing a wound, the method comprising:
obtaining a biocompatible wound dressing, comprising:
a biocompatible interface; and
multiple fixation elements that are disposed on the biocompatible interface, wherein the fixation elements are configured to couple the biocompatible interface to a subcutaneous tissue on an undersurface of skin when the fixation elements are inserted below the subcutaneous tissue; and
placing the biocompatible wound dressing inside a wound such that the fixation elements couple the biocompatible interface to the subcutaneous tissue.

19. The method of claim 18, further comprising applying an additional dressing to the wound, wherein the additional dressing is coupled to at least one of: (i) a suction device that provides negative pressure wound therapy to the wound and (ii) a mechanical closure device.

20. The method of claim 18, wherein the biocompatible interface and the fixation elements are each configured to at least one of: (i) absorb into a tissue space and (ii) incorporate into the tissue space after prolonged use of the biocompatible wound dressing in the tissue space.
